(19) United States
(12) Patent Application Publication
(10) Pub. No.: US 2018/0000445 A1
(43) Pub. Date: Jan. 4, 2018

(54) Systems and Methods for Lesion Formation Feedback

(71) Applicant: St. Jude Medical, Cardiology Division, Inc., St. Paul, MN (US)

(72) Inventors: John W. Sliwa, San Jose, CA (US); Zhenyi Ma, Santa Clara, CA (US)

(21) Appl. No.: 15/545,487

(22) PCT Filed: Jan. 4, 2016
(86) PCT No.: PCT/US2016/012031

§ 371 Date: Jul. 21, 2017

Related U.S. Application Data
(60) Provisional application No. 62/113,833, filed on Feb. 9, 2015.

(51) Int. Cl. A61B 8/08; A61B 8/12; A61B 8/00

(52) U.S. Cl. CPC A61B 8/085; A61B 8/543; A61B 8/5223; A61B 8/0883; A61B 8/445; A61B 8/12; A61B 8/485

(57) ABSTRACT

Apparatuses, systems, and methods of monitoring lesion formation using one-dimensional echograms are disclosed. In certain aspects, lesion formation progress is monitored using the intensity of reflectors in successive echograms during ablation. In another aspect, lesion formation progress is monitored based upon actual or apparent movement of acoustic reflectors before and after ablation. In still another aspect, the presence or absence of resonant microbubbles known to populate forming lesions are used to provide feedback on lesion formation. A lesion analysis processor can be programmed to determine lesion formation progress using any of the foregoing approaches, either alone or in various combinations.

### CROSS-REFERENCE TO RELATED APPLICATIONS

This application claims the benefit of U.S. provisional application No. 62/113,833, filed 9 Feb. 2015, which is hereby incorporated by reference as though fully set forth herein.

### BACKGROUND

The instant disclosure relates generally to tissue ablation. In particular, the instant disclosure relates to systems, apparatuses, and methods for monitoring the formation of a lesion, for example in cardiac tissue, using echography,

Catheters are used in a variety of diagnostic and therapeutic procedures, for example to diagnose and/or treat conditions such as atrial arrhythmias, For example, a catheter carrying one or more electrodes can be deployed and manipulated through a patient's vasculature and, once located at the intended site, radiofrequency (“RF”) energy can be delivered through the electrodes to ablate tissue.

In some catheters, an additional sensor, such as an ultrasound sensor, is provided in the catheter tip to provide additional information during the primary diagnosis or therapy. For example, practitioners often desire information about lesion formation, such as lesion depth, lesion pop-potential, and lesion transmurality. Thus, RF ablation catheters can include one or more ultrasound sensors, located within the hollow tip of the catheter, that can be used to monitor the progress of a lesion forming in the tissue being treated and/or to confirm one or more characteristics of the lesion once created.

### BRIEF SUMMARY

Disclosed herein is a method of measuring lesion formation in a tissue, including: acquiring a first echogram scanline of the tissue from an ultrasound imaging device operating at a first transmit power and a first gain, wherein the first echogram scanline includes a first line scan of the tissue from a surface of the tissue to a depth within the tissue; delivering an increment of ablation to the tissue; acquiring a second echogram scanline of the tissue from the ultrasound imaging device after acquiring the first echogram scanline and delivering the increment of ablation, wherein the second echogram scanline includes a second line scan of the tissue from the surface of the tissue to the depth within the tissue; identifying a region that appears darker in the second echogram scanline than in the first echogram scanline when the second echogram scanline is acquired with the ultrasound imaging device operating at the first transmit power and the first gain; increasing a brightness of the second echogram scanline until the region that appears darker in the second echogram scanline than in the first echogram scanline appears as bright in the second echogram scanline as it appears in the first echogram scanline; and, after increasing the brightness of the second echogram scanline: identifying a region that appears brighter in the second echogram scanline than it appears in the first echogram scanline; and providing feedback about a lesion forming in the tissue based upon the region that appears brighter in the second echogram scanline than it appears in the first echogram scanline. The first echogram scanline and the second echogram scanline can both be A-line scan echograms along the same, or a substantially similar, beam path through the tissue (e.g., by orienting the ultrasound imaging device at a common orientation relative to the tissue when acquiring both the first and second echogram scanlines). Likewise, it is contemplated that the first and second echogram scanlines can be acquired at a common time point in the cardiac cycle (e.g., at common cardiac deformation states). The brightness (that is, the received echo amplitude) can be increased by increasing a receive gain of the ultrasound imaging device relative to the first gain prior to acquiring the second echogram scanline and/or by increasing transmit power of the ultrasound imaging device relative to the first transmit power prior to acquiring the second echogram scanline.

The step of providing feedback about a lesion forming in the tissue based upon the region that appears brighter in the second echogram scanline than it appears in the first echogram scanline can include providing lesion depth information according to a depth of the region that appears brighter in the second echogram scanline than it appears in the first echogram.

In another embodiment, a method of measuring lesion formation in cardiac tissue, includes: acquiring a first A-line scan echogram of the cardiac tissue at a first cardiac deformation state; acquiring a second A-line scan echogram of the cardiac tissue at a second cardiac deformation state; computing a baseline elasticity of the cardiac tissue from the first A-line scan echogram and the second A-line scan echogram; ablating the cardiac tissue; and, after ablating the cardiac tissue: acquiring a third A-line scan echogram of the cardiac tissue at the first cardiac dethrmation state; acquiring a thurth A-line scan echogram of the cardiac tissue at the second cardiac deformation state; computing a revised elasticity of the cardiac tissue from the third A-line scan echogram and the fourth A-line scan echogram; and providing feedback about a lesion forming in the cardiac tissue based upon the revised elasticity of the cardiac tissue. For example, feedback about a lesion forming in the cardiac tissue can be provided based upon a comparison of the revised elasticity to the baseline elasticity. Alternatively or additionally, the feedback can be provided based upon a comparison of the revised elasticity to a desired elasticity.

In aspects, computing a baseline elasticity of the cardiac tissue includes computing a baseline elasticity of the cardiac tissue based upon movement of acoustic reflectors within the cardiac tissue between the first A-line scan echogram and the second A-line scan echogram, while computing a revised elasticity of the cardiac tissue includes computing a revised elasticity of the cardiac tissue based upon movement of acoustic reflectors within the cardiac tissue between the third A-line scan echogram and the fourth A-line scan echogram.

In yet another embodiment, a method of measuring lesion formation in cardiac tissue, includes: acquiring a first A-line scan echogram of the cardiac tissue; ablating the cardiac tissue; and, after ablating the cardiac tissue: acquiring a second A-line scan echogram of the cardiac tissue; determining an apparent shrinkage of the cardiac tissue from the first A-line scan echogram and the second A-line scan echogram; and providing feedback about a lesion forming in the cardiac tissue based upon the apparent shrinkage of the cardiac tissue. It is desirable for the first A-line scan echogram and the second A-line scan echogram to be acquired with the cardiac tissue in a first cardiac deformation state.

It is contemplated that the apparent shrinkage of the cardiac tissue will be determined based upon apparent movement of acoustic reflectors within the cardiac tissue between the first A-line scan echogram and the second A-line scan echogram.

In still another embodiment, a method of measuring lesion formation in cardiac tissue includes: acquiring a first A-line scan echogram of the cardiac tissue at a first cardiac deformation state; ablating the cardiac tissue; and, after ablating the cardiac tissue; acquiring a second A-line scan echogram of the cardiac tissue at the first cardiac deformation state; and providing feedback about a lesion forming in the cardiac tissue by analyzing at least one of actual movement of acoustic reflectors within the cardiac tissue and apparent movement of acoustic reflectors within the cardiac tissue using at least the first A-line scan and movement the second A-line scan. For example, the feedback can use apparent movement of acoustic reflectors due to increases in acoustic velocity within the cardiac tissue. In other aspects, the feedback uses actual movement of acoustic reflectors due to decreases in tissue elasticity within the cardiac tissue.

Also disclosed herein is a method of measuring lesion formation in a tissue, including: emitting narrowband pulsed acoustic energy towards the tissue at a preset frequency, wherein the preset frequency corresponds to a resonant frequency of a microbubble characteristic of lesion formation; detecting echoes of the emitted acoustic energy; and providing feedback about a lesion forming in the tissue by analyzing a distribution of the microbubble characteristic of lesion formation within the tissue using the detected echoes of the emitted acoustic energy. It is contemplated that the method will also include: ablating the tissue; and, after ablating the tissue, repeating the steps of: emitting narrowband pulsed acoustic energy towards the tissue at a preset frequency; detecting echoes of the emitted acoustic energy; and analyzing a distribution of the microbubble characteristic of lesion formation within the tissue using the detected echoes of the emitted acoustic energy at the resonant frequency and/or a harmonic thereof, wherein providing feedback about a lesion forming in the tissue includes analyzing a change in the distribution of the microbubble characteristic of lesion formation within the tissue from prior to ablating the tissue to after ablating the tissue.

In still another embodiment, a system for measuring lesion formation in a tissue includes a lesion analysis processor programmed to receive as input at least two A-line scan echograms of the tissue, to determine progress of a lesion forming in the tissue from the at least two A-line scan echograms, and to output feedback about the lesion. The lesion analysis processor can determine the progress of the lesion forming in the tissue using one or more approaches described herein (e.g., changes in brightness from echogram to echogram; changes in tissue elasticity as evidenced by changes in acoustic reflector movement between echograms; tissue shrinkage as evidenced by acoustic reflector movement between echograms; changes in resonant microbubble distribution from echogram to echogram).

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts an ablation and lesion feedback system including an exemplary catheter including a sensor-bearing tip.

FIG. 2 is a transverse cross-section of a sensor-bearing catheter tip according to a first aspect disclosed herein. Portions of the tip are rendered transparent in order to illustrate details of the interior thereof.

FIG. 3 is a perspective view of a sensor-bearing catheter tip according to a second aspect disclosed herein. Portions of the tip are rendered transparent in order to illustrate details of the interior thereof.

FIG. 4 is a perspective view of a sensor-bearing catheter tip according to a third aspect disclosed herein. Portions of the tip are rendered transparent in order to illustrate details of the interior thereof.

FIG. 5 is a perspective view of a sensor-bearing catheter tip according to a fourth aspect disclosed herein. Portions of the tip are rendered transparent in order to illustrate details of the interior thereof.

FIG. 6 is a perspective view of a sensor-bearing catheter tip according to a fifth aspect disclosed herein. Portions of the tip are rendered transparent in order to illustrate details of the interior thereof.

FIG. 7 schematically illustrates the formation of a lesion.

FIG. 8 is a flowchart of representative steps that can be carried out to monitor the formation of a lesion according to a first method disclosed herein.

FIGS. 9a and 9b illustrate the application of the lesion formation monitoring method represented by FIG. 8.

FIG. 10 is a flowchart of representative steps that can be carried out to monitor the formation of a lesion according to a second method disclosed herein.

FIG. 11 is a flowchart of representative steps that can be carried out to monitor the formation of a lesion according to a third method disclosed herein.

FIG. 12 is another schematic illustration of the formation of a lesion.

FIG. 13a illustrates a narrowband acoustic pulse that can be utilized to monitor the formation of a lesion according to a fourth method disclosed herein.

FIG. 13b illustrates the acoustic echoes resulting from the narrowband acoustic pulse shown in FIG. 13a when applied in the arrangement depicted in FIG. 12.

### DETAILED DESCRIPTION

The present disclosure provides methods, apparatus, and systems for monitoring the formation of a lesion in tissue. For purposes of illustration, several exemplary embodiments will be described herein in detail in the context of a radiofrequency (“RF”) ablation catheter including an acoustic sensor (e.g., a pulse-echo transducer; a photoacoustic transducer) that can be used to monitor the progress of the lesion being formed in an adjacent tissue. It should be understood, however, that the methods, apparatuses, and systems described herein can be utilized in other contexts.

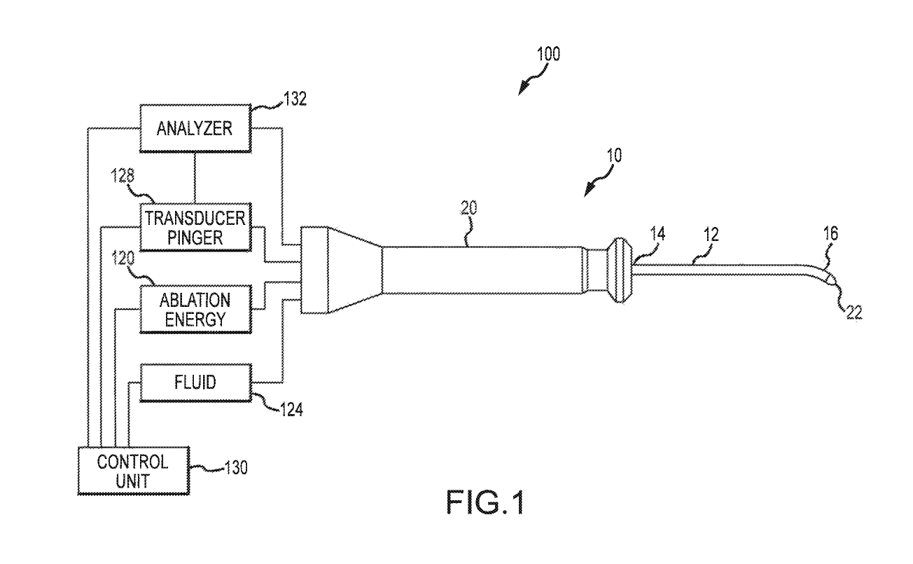

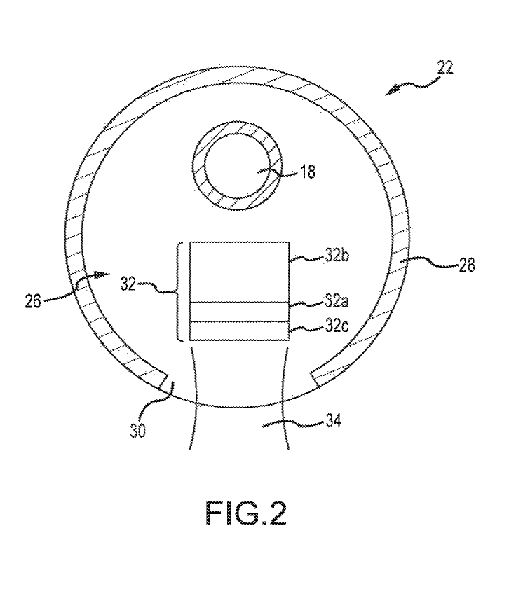

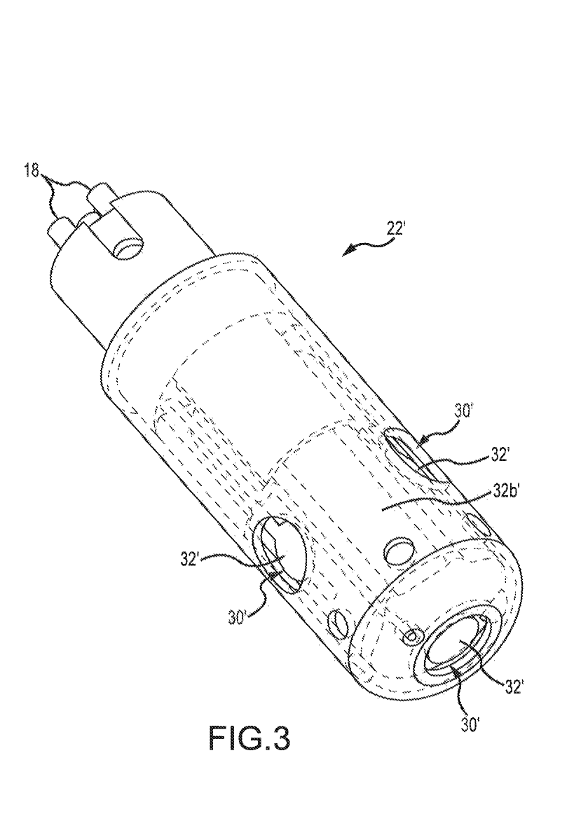

FIG. 1 is a schematic diagram of an ablation and lesion feedback system 100 including an exemplary catheter 10. As shown in FIG. 1, catheter 10 generally includes an elongate, hollow, and flexible tubular body 12 having a proximal end 14 and a distal end 16. Tubular body 12 defines a lumen 18 (not visible in FIG. 1, but visible, inter alia, in FIG. 2). Although only a single lumen 18 is depicted in certain figures for clarity of illustration, it should be understood that any number of lumens 18 can be used without departing from the scope of the instant teachings (see, e.g., multiple lumens as depicted in FIG. 3). As the person of ordinary skill in the art will appreciate, tubular body 12 can also contain electrical interconnect wires, pull-wires, thermocouple leads, and the like.

Proximal end 14 of tubular body 12 is attached to a catheter control handle 20. Catheter control handle 20 can include, for example, an actuator (not shown) coupled to suitable structure (e.g., pull wires and/or pull rings) within tubular body 12 in order to effect the deflection of distal end 16 in one or more bending planes. It can also include electrical power and/or signal connections to additional components of ablation system 100 as discussed in further detail below.

A hollow tip 22 is attached to distal end 16 of tubular body 12. As used herein, the term “hollow” means that tip 22 includes at least one cavity, for example to contain ultrasound transducer 32 as described below. Various suitable embodiments of tip 22 will be described in further detail below with reference to FIGS. 2-6. In general, however, for purposes of the instant disclosure, tip 22 can include an RF ablation element, such as a tip electrode, and can, as such, be connected with an ablation energy source 120, such as an RF generator.

FIG. 2 is a cross-sectional view of hollow tip 22 according to certain aspects of the instant disclosure. As shown in FIG. 2, an irrigant lumen 18 of tubular body 12 is in fluid communication with the interior 26 of hollow tip 22, which is defined by a wall 28 of hollow tip 22. An irrigant (e.g., saline) or other fluid can be delivered from fluid source 124 (shown in FIG. 1), through lumen 18, and into hollow tip 22, for example for cooling purposes, for energy transmission purposes, and/or for acoustic matching purposes.

Wall 28 of hollow tip 22 further includes a window (or “beam hole”) 30 (e.g., a break in wall 28). Window 30 allows for the passage of acoustic energy to and/or from a sensor, such as pulse/echo ultrasound transducer 32 disposed within interior 26 of hollow tip 22, along an acoustic beam path 34. As seen in FIG. 2, and as will be familiar to the person of ordinary skill in the acoustic art, transducer 32 can include a piezomaterial layer 32a, an attenuative backer material 32b, and one or more acoustic matching layers 32c. It should also be understood that, as used herein, the term “transducer” encompasses all manner of ultrasound transducers, including, without limitation, single element transducers, multi-element transducers, focused transducers, unfocused transducers, transducers with acoustic matching layers 32c, transducers without acoustic matching layers 32c, piezomaterial-based (e.g., PZT, PVDF) transducers, photoacoustic-based transducers, thermoacoustic based transducers, and capacitive micromechanical ultrasound transducers (“CMUT”).

Window 30 also allows for irrigant to pass out of interior 26 of hollow tip 22, for example for tissue and transducer cooling purposes, acoustic energy transmission purposes, and/or for ablation energy coupling to adjacent tissue. The irrigant, such as saline, can benefit the coupling and transmission of both RF ablation energy and pulse-echo acoustic energy to (and, in the case of pulse-echo energy, from) the adjacent tissue.

A transducer pinger 128 (see FIG. 1), which might have more than one transducer channel, supplies pinging energy, such as electrical energy pulses, to transducer 32, resulting in the generation of an acoustic wave along beam path 34 as shown in FIG. 2. A control unit 130 (also shown in FIG. 1) is provided for controlling the ablation and the acoustic pinging during ablation. For instance, control unit 130 can be configured to carry out duty cycling or synchronization for both ablation and pinging. (The term “pinging” is used herein to mean transmitting acoustic energy and then receiving the reflected or echoed acoustic energy.)

An acoustic pinger echo analyzer or acoustic receiver 132 is provided to condition and analyze the data collected by transducer 32 to provide lesion feedback. The information can be presented to a practitioner (e.g, using a graphical user interface) to provide real-time assessment of the ablation target, the ablation process, and/or the ablation result. The information may additionally or alternatively be used by the system itself without operator intervention, for example as input to a feedback control loop controlling ablation power and/or irrigant cooling to avoid steam pops and/or to achieve a desired lesion depth.

Thus, one aspect disclosed herein is directed to an RF ablation catheter with one or more ultrasound transducers therein or thereon, wherein the transducer is capable of, inter alia, acoustic lesion feedback. The catheter is capable of delivering an RF ablating tip to a patient's tissue to be ablated. These aspects and others are described in U.S. patent application publication no. 2012/0265069, which is hereby incorporated by reference as though fully set forth herein.

FIG. 3 illustrates another embodiment of a tip, designated 22′, suitable for use in connection with the teachings herein. Tip 22′ includes four transducers 32′ arranged in what can be referred to as a “3+1” arrangement. Three such transducers 32′ are oriented with their beam paths (and corresponding windows 30′) at about 90 degrees to the longitudinal axis of tip 22′ (e.g., side-looking) and at about 120 degrees from each other about the circumference of tip 22′ (only two of the side-looking transducers 32′ are visible in FIG. 3, with the third side-looking transducer 32′ on the underside of hollow tip 22′). The fourth transducer 32′ is oriented with a forward axially-directed beam path (and corresponding window 30′) substantially parallel to the longitudinal axis of tip 22′ (e.g., forward-looking).

Transducers 32′ in tip 22′ can be attached to a common attenuative backer 32b′. Backer 32b′ can be made of an injection-molded polymer containing tungsten particles; the polymer can be, for example, an amorphous thermoplastic polyetherimide (e.g., Ultem™), poly ether sulfone (“PES”), or another strong, creep-resistant, high-temperature polymer material such as poly ether ether ketone (“PEEK”). Injection molding at high pressure and temperature can advantageously utilize molding pellets that already contain the desired concentration of tungsten particles to achieve the desired acoustic attenuation and acoustic impedance of backer 32b′. Moreover, the high-temperature, high-strength nature of the polymer allows for transducer lamination and for some deposited or laminated electrodes to be deposited on backer 32b′ at modest processing temperatures.

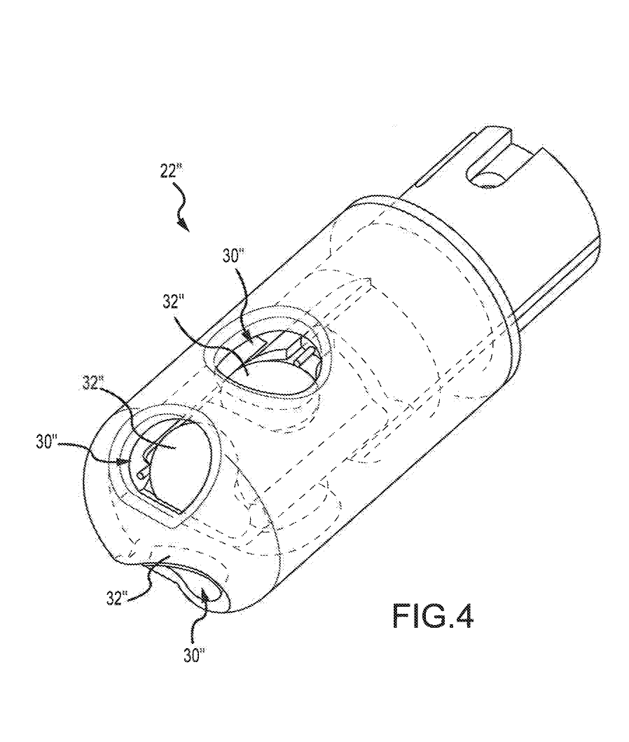

FIG. 4 is yet another embodiment of a tip 22″ suitable for use in connection with the teachings herein. Tip 22″ likewise includes four transducers 32″ arranged in what can be referred to as a “2+2” arrangement. Two transducers 32″ are oriented with their beam paths (and corresponding windows 30″) at about 45 degrees to the longitudinal axis of tip 22″; they are therefore partially forward-looking and partially side-looking (referred to herein as “forward/side-looking”). The other two transducers 32″, which can be oriented proximally of the forward/side-looking transducers 32″, are oriented to be primarily side-looking, and can be spaced about 180 degrees from each other about the circumference of hollow tip 22″.

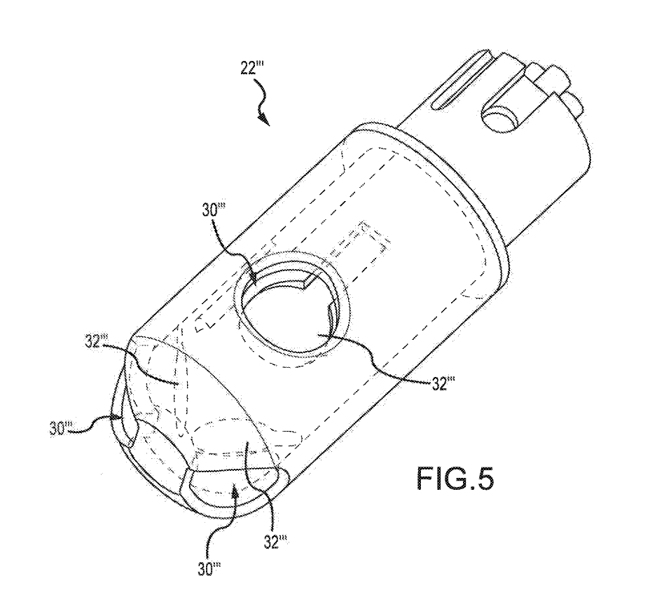

A variation on the 2+2 arrangement is shown as tip 22′″ in FIG. 5. In FIG. 5, the forward/side-looking transducers 32′″ are rotated about 90 degrees about the longitudinal axis of hollow tip 22′′ relative to the orientation shown in FIG. 4. Thus, in tip 22′, each of the forward/side-looking transducers 32′″ has a beam path in a unique plane, whereas, in the embodiment of FIG. 4, all four transducers 32″ have their beam paths in a common plane.

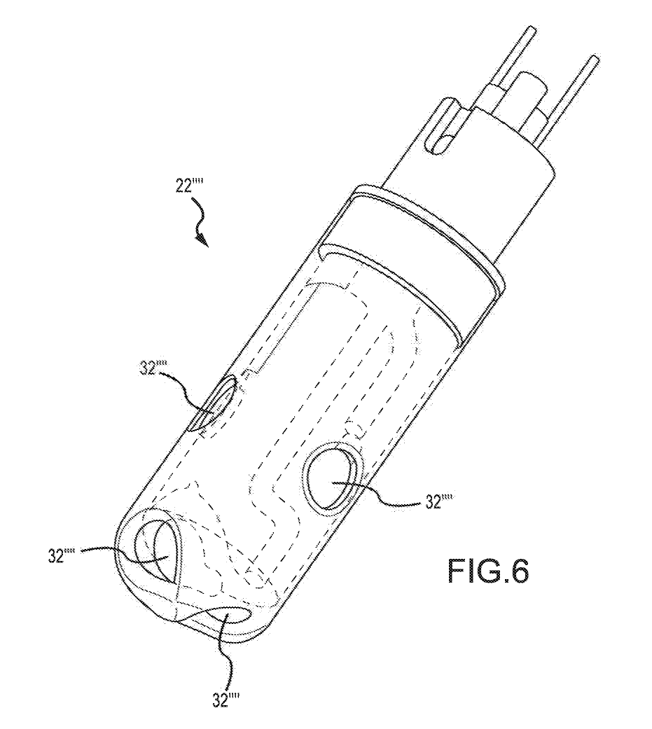

A further embodiment of a tip, designated 22″″, is shown in FIG. 6. In tip 22″″, there are five transducers 32″″ oriented in what can be referred to as a “3+2” arrangement. In particular, two transducers 32″ are oriented to be forward/side-looking, while three transducers 32″″ are oriented to be side-looking.

It should be understood that the embodiments depicted in FIGS. 4-6 can likewise utilize a common attenuative backer analogous to backer 32b′ of FIG. 3.

Other arrangements are also contemplated. For example, the side-looking transducers can be removed from the configurations shown in FIGS. 4-6, leaving only the forward/side-looking transducers.

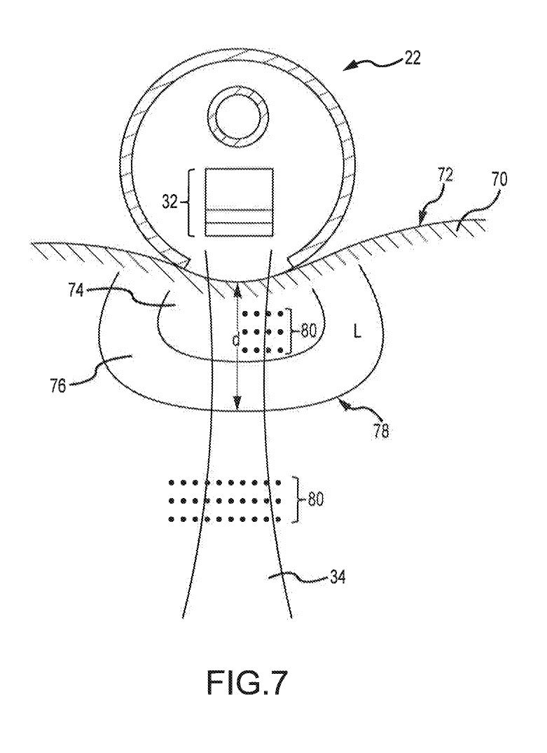

FIG. 7 depicts a lesion (denoted “L”) forming in a tissue 70, such as a cardiac tissue, for example as a result of RF energy delivered by catheter 12. As those of ordinary skill in the art will appreciate, lesion L will typically include a core region 74, which is slightly browned, and a surrounding reuion 76, which is white upon sectioning. Lesion L is defined by a lesion front 78, which extends a depth “d” below tissue surface 72. Although lesion front 78 will advance through tissue 70 as additional RF energy is applied, at any given time, tissue beyond lesion front 78 is substantially unaffected (e.g., it has its inherent naturally high elasticity and low ultrasonic reflectivity).

Transducer 32 can be used to monitor the formation of lesion L according to various methods and aspects disclosed herein. In general, these aspects compare echograms acquired at different times (e.g., before any ablation, after some incremental ablation, after ablation is fully complete) to measure the formation of lesion L. It is also desirable for the echograms to extend depthwise from the tissue surface 72 to a depth beyond the desired final depth d of lesion L, such that an unablated region remains “underneath” (that is, below or behind) the desired lesioned region L in the echogram. Although several embodiments are disclosed herein in connection with A-line scan echograms, other ultrasound imaging modes may be employed in other embodiments to analyze tissue 70.

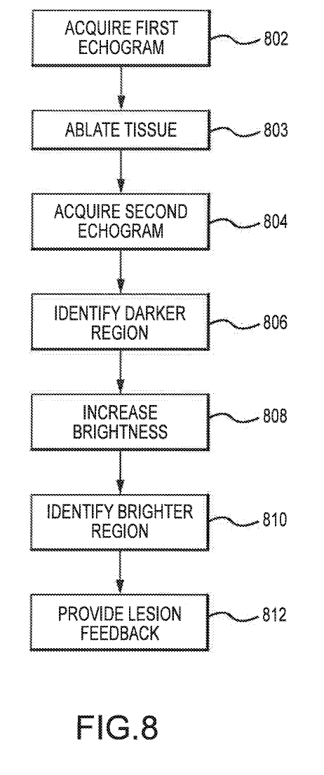

FIG. 8 is a flowchart 800 of representative steps that can be carried out to measure the formation of lesion L according to a first aspect of the disclosure. In block 802, a first echogram (e.g., a first A-line scan) of tissue 70 is acquired, and optionally saved, for example using transducer 32 operating at a first transmit power and a first gain.

In block 804, a second echogram (e.g., a second A-line scan) of tissue 70 is acquired at a later time, for example following at least some incremental ablation of tissue 70 in block 803. As used herein, the term “incremental ablation” means that the final overall depth and/or volume of lesion L is formed in discrete depth and/or volume increments, allowing for the teachings herein to be applied between increments, for example to monitor lesion progress and/or adjust power, time, and/or other operating parameters for future ablation increments. An incremental ablation as disclosed herein can be very fine (e.g., 100 or more increments to create the full depth and/or volume)or very coarse; indeed, it can even be completed in a single increment.

Like the first echogram, the second echogram can be acquired using transducer 32 and optionally saved. It is also desirable for the second echogram to be acquired with tip 22 in substantially the same position and rotational orientation with respect to the beating heart as it was in when acquiring the first echogram, as can be done by synchronizing the pinging of transducer 32 with the heartbeat, so that both echograms capture substantially the same scan line through tissue 70.

As those of ordinary skill in the art will appreciate, as lesion L is formed, an echogram of tissue 70 can exhibit brighter or higher-amplitude contrast due, inter glia, to microbubbling and tissue protein crosslinking in region L, particularly at near-field depths less than d. The growing microbubble cloud at more superficial depths, however, can prevent ultrasound from penetrating tissue 70 more deeply, which leads to the deeper tissue appearing darker in echograms taken after lesioning (e.g., the second echogram) than in echograms taken before lesioning (e.g., the first echogram). In other words, the formation of microbubbles due to heated tissue at shallower depths masks the cooler tissue's own inherent brightness or contrast at deeper depths. This darkened, unablated and unbubbled region is identified in block 806. The ablation practitioner will also appreciate that, although much of the darkening of deeper tissue is due to nearer-surface microbubbles, some of it is attributable to protein crosslinking in the hot tissue.

In block 808, the brightness (e.g., the amplitude gain) of the second echogram is increased to restore the original brightness of the darkened unablated deeper region. As used herein, the term “brightness” refers to the amplitude of the received echo from a given point in tissue 70, such as at a particular depth along a particular scan line at such an unablated depth greater than d. For example, the brightness or echo amplitude can be increased at all depths by increasing the logarithmic signal gain amplitude of the second echogram or by operating transducer 32 at a higher transmit power and/or with an increased gain relative to the first echogram until the deeper, darkened, unlesioned tissue is restored to its original brightness or reflective amplitude. Increasing the transmit power of transducer 32 may be somewhat more desirable than increasing gain, as increasing gain can introduce noise into the acquired echogram. In the interest of time, coarse adjustments to transmit power and/or gain can be made prior to acquiring the second echogram based on experience.

When the deeper, darkened unablated region of the second echogram is restored to its original preablation brightness the brightness or echo-amplitude versus depth of the first echogram), the brightness of the more superficial region of the second echogram will also increase, ultimately appearing brighter in the second echogram than the first echogram. This brighter region is identified in block 810.

In block 812, the brighter-depth region identified in block 810 is used to provide feedback about lesion L forming in tissue 70. The brighter region will typically start at or near tissue surface 72 and extend to tissue that has no microbubbling and no crosslinking (i.e., tissue that is unburned and not very hot). For example, in some embodiments, the depth of the brighter region can be provided to a user directly as the depth d of lesion L. In other embodiments, the depth d of lesion L can be extrapolated from the depth of the brighter region, for example by multiplying the depth of the brighter region by a correction factor that can be experimentally determined for a given transducer and/or operating frequency.

The foregoing steps can also be repeated, either at the same location as lesion L continues to form incrementally, or at a different location. In the case where the foregoing steps are repeated, the later-acquired echogram can be compared to the originally-acquired echogram, the immediate prior echogram, or to a composite or average thereof.

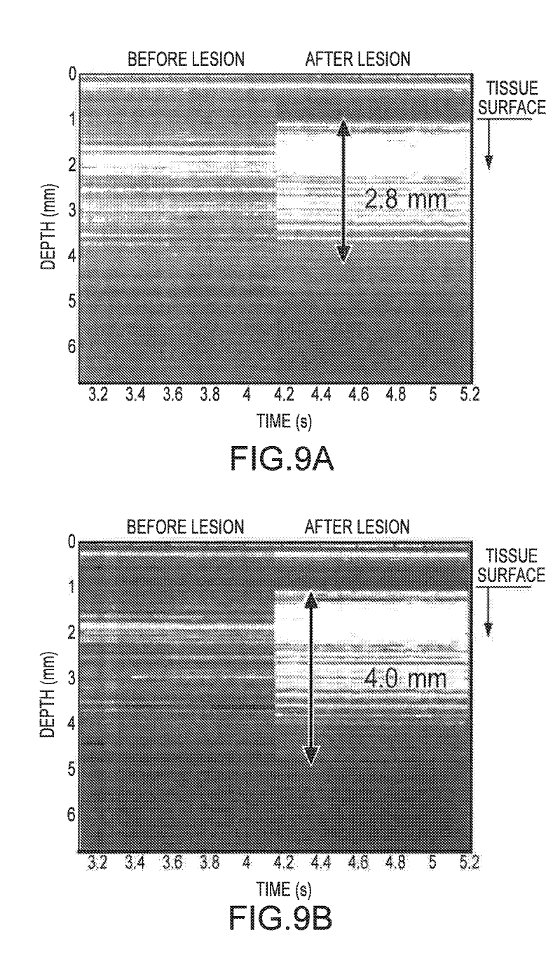

FIGS. 9a and 9b are A-line scan echograms that illustrate the use of the method described above to identify the depth of a lesion. Starting from the left-hand side of each figure (around 3 seconds) are quick echograms taken prior to any ablation. The right-hand side of each figure (starting around 4.2 seconds) is a post-ablation quick echogram in the same tissue location. In FIG. 9a, one can see the darkened region described above prior o any brightness adjustment. In FIG. 9b, the darkened region has been restored to its previous (i.e., pre-ablation) brightness, which has also increased the brightness of more superficial regions relative to the pre-ablation echogram. This region, which extends about 4.0 mm from the tissue surface, corresponds closely to the depth of the lesion when the same is examined in a photomicrograph.

In addition to masking underlying, non-lesioned tissue, the ordinarily skilled artisan will also appreciate that lesioned tissue is harder than non-lesioned tissue. As such, lesioned tissue deforms less under mechanical loads, such as tissue loading occurring naturally during the heartbeat cycle. With reference to FIG. 7, for example, core region 74 will be harder than surrounding region 76, and surrounding region 76 will be harder than those portions of tissue 70 beyond lesion front 78. There are known techniques, including two- and three-dimensional elastography, to utilize this characteristic to monitor lesion formation. In some embodiments disclosed herein, these techniques are applied in only one dimension, obtaining elastographic stiffness only along that available beam direction.

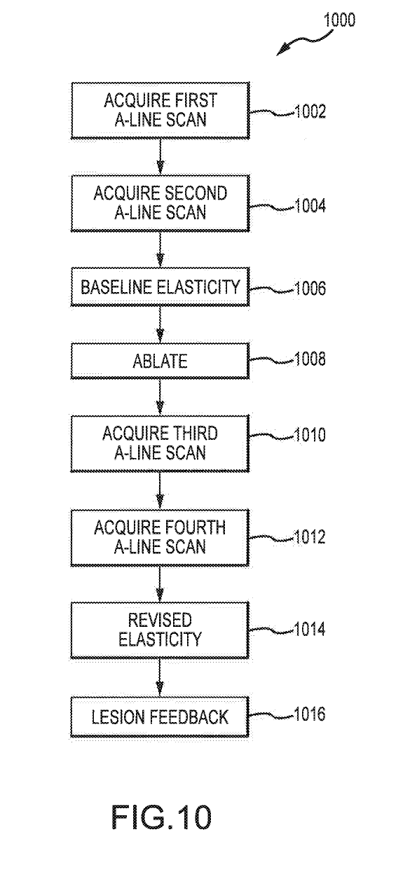

FIG. 10 is a flowchart 1000 of representative steps that can be carried out to use this same elastographic characteristic to measure lesion formation using one-dimensional echograms (e.g., A-line scans synchronized to the heartbeat to be along the same tissue path) by tracking the movement of acoustic reflectors 80, such as tissue features and/or stable microbubbles, within tissue 70 along beam path 34. In particular, the movement of acoustic reflectors 80 within tissue 70 along beam path 34 due to the heartbeat flexing of the heart wall can be used as a measure of lesion formation. Because the harder lesioned tissue will deform less than non-lesioned (or less-lesioned) tissue during the heartbeat cycle, the acoustic reflectors 80 within those regions will move less relative to each other along beam path 34. Although only a small number of uniformly distributed acoustic reflectors 80 are shown in FIG. 7, it should be understood that this is only for the sake of illustration and that acoustic reflectors 80 will more typically be more widespread and randomly distributed through tissue 70, and in both ablated and unablated regions.

In block 1002, a first A-line scan echogram of tissue 70 is acquired. The point in the heartbeat cycle when this first echogram is acquired is referred to herein as the “first cardiac deformation state.”

In block 1004, a second A-line scan echogram of tissue 70 is acquired. This second echogram is desirably acquired at a different point in the heartbeat cycle, referred to herein as the “second cardiac deformation state.” It is advantageous for the first and second cardiac dethrmation states to be as different as possible (e.g., maximum systole vs. maximum diastole) in order to maximize the relative deformation of tissue 70, and thus the movement of specific reflectors 80, between the two states.

A baseline tissue elasticity along the direction of beam path 34 is determined in block 1006 from the movement of acoustic reflectors 80 along beam path 34 between the first and second echograms acquired at the first and second cardiac deformation states before ablation stiffens any tissue.

Ablation, such as at least some incremental ablation, is carried out in block 1008. In blocks 1010 and 1012, after at least some ablation has occurred, a second, post-ablation pair of echograms is acquired, again taken at the first and second cardiac deformation states. These two new echograms, referred to herein as the third and fourth echograms, respectively, allow a revised elasticity along beam path 34 to be determined in block 1014, once again from the movement of acoustic reflectors 80 along beam path 34 between the first and second cardiac deformation states post-ablation. The ablation will typically reduce the movement of acoustic reflectors 80 relative to tip 22 due to reduced tissue elasticity.

In block 1016, feedback regarding the formation of lesion L is provided based upon the revised elasticity determined in block 1014. In certain aspects, the feedback is based upon a comparison of the revised elasticity to the baseline elasticity. In other aspects, the feedback is based upon a comparison of the revised elasticity to a desired elasticity that is known to correspond to the formation of a desired lesion.

It is contemplated that the foregoing steps can be repeated, either at the same location as lesion I, continues to form, or at a different location. In the case where the foregoing steps are repeated, the revised elasticity can be compared to the baseline elasticity, the immediate prior revised elasticity (that is, each revised elasticity can be used as a new baseline elasticity for subsequent measurements), or to a composite or average thereof.

The ordinarily skilled artisan will also appreciate that lesions cause significant and long-lasting omnidirectional tissue shrinkage due, for example, to cross-linking and desiccation. This component of shrinkage can be detected by tracking acoustic reflectors 80 along just one direction. In addition to this actual physical shrinkage, however, there are at least two factors that will result in an echogram depicting apparent shrinkage in lesioned tissue. First, because the lesioned tissue is hotter than surrounding tissue, the speed of sound therethrough will be higher than the value assumed when taking echograms, usually about 1440 m/s. Second, because the lesioned tissue is harder than surrounding tissue, the speed of sound therethrough will be higher than the value assumed when taking echograms.

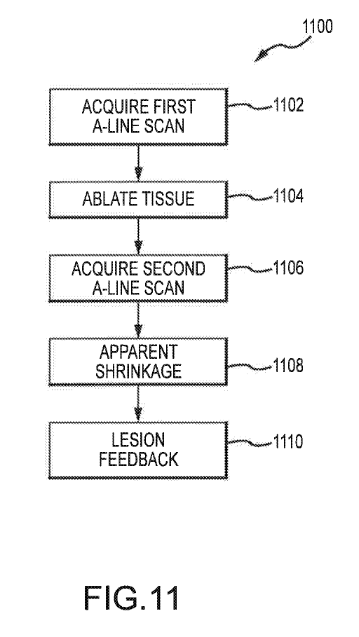

FIG. 11 is a flowchart 1100 of representative steps that can be carried out to use this apparent shrinkage to measure lesion formation using one-dimensional echograms (e.g., A-line scans). As with the embodiment discussed above in connection with FIG. 10, this can be carried out by tracking the movement of acoustic reflectors 80 within tissue 70 along beam path 34. In the case of the embodiment of FIG. 11, however, it is permanent shrinkage relative to a starting tissue thickness, which should be accompanied by reduced tissue elasticity, that is of interest. Thus, in connection with the method depicted in FIG. 11, it is desirable for all echograms to be acquired at the same cardiac deformation state and for the tissue temperature to be approximately the same between A-line pinging scans.

In block 1102, a first A-line scan echogram of tissue 70 is acquired. Ablation, such as at least some incremental ablation, is carried out in block 1104. Following ablation, a second A-line scan echogram of tissue 70 is acquired in block 1106; as discussed above, the second A-line is desirable acquired at the same cardiac state, for example by synchronizing the transducer pinging to the heartbeat. In block 1108, the apparent shrinkage is determined from the apparent movement of acoustic reflectors 80 between the first and second echograms, Lesion feedback is provided in block 1110. It is contemplated that this shrinkage-based analysis can be done in parallel with the elasticity-based analysis discussed above to provide dual indications of the formation of lesion L.

Of course, the steps illustrated in FIG. 11 can be repeated, either at the same location as lesion L continues to form, or at a different location. In the case where the foregoing steps are repeated, and unlike the embodiments previously described, apparent shrinkage will typically be determined relative to the initial (pre-lesioning) state of no apparent shrinkage, rather than relative to an immediately previous apparent shrinkage measurement, a composite or average, or the like.

In yet another aspect, lesion formation can be measured by monitoring resonant microbubbles. It is known that a forming lesion, as well as adjacent tissue, is populated by nucleating and nucleated microbubbles. These microbubbles include gas that was previously in solution; at higher temperatures, they can include water vapor and steam. The radius r of a microbubble determines its resonant frequency f_{f} under ultrasonic excitation according to the equation

fᵣ=1/2πr\sqrt{Sa*b*β/m},

where Sa is the microbubble adiabatic stiffness, b is the reciprocal of the polytropic coefficient, β is the surface tension coefficient, and m is the mass of the system. Acoustic practitioners will appreciate that microbubbles typically also have harmonic resonance modes; it is contemplated that excitation can occur at a first frequency, with echoes received at a lower or higher frequency harmonic of the excitation frequency.

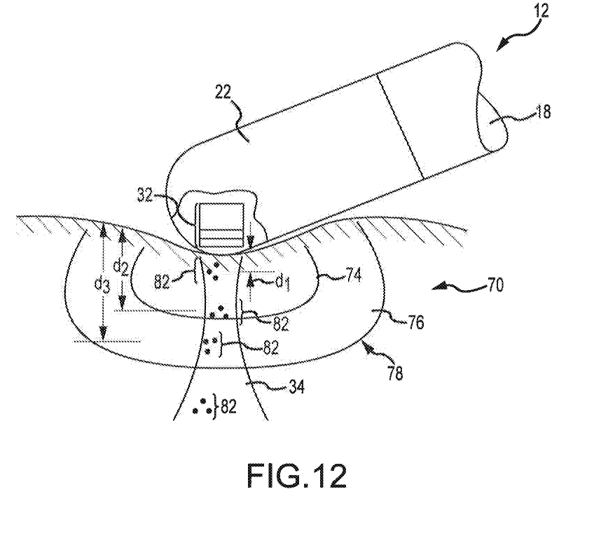

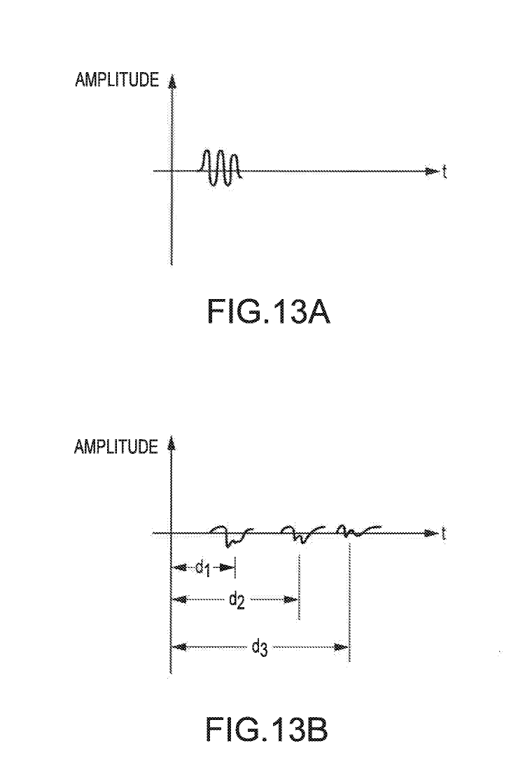

FIG. 12 depicts microbubbles 82 forming in tissue 70 as lesion L forms. To detect these microbubbles, transducer 32 is operated to emit narrowband pulses, for example as shown in FIG. 13a. The frequency of these pulses can be preset to correspond to the resonant frequency fᵣ of certain radius microbubbles that are known (or, in embodiments, experimentally determined) to be characteristic of lesion formation and that excite the above-described harmonics. For example, the frequency of the narrowband pulses can be preset to correspond to the resonant frequency of microbubbles that are known to occur in a typical lesion resulting from a given ablation power, irrigation depth, and time. As discussed above, the microbubbles may respond at the excitation resonant frequency or at a lower or higher harmonic thereof.

It is also contemplated that, rather than using a fixed narrowband excitation frequency, a frequency ramped narrowband signal wherein several measurements, each at a different narrowband ramped frequency, can be employed.

FIG. 13b is a representative returned echo plot of the narrowband pulses shown in FIG. 13a for the arrangement depicted in FIG. 12. Three echoes are shown, corresponding to the microbubbles 82 at depths d₁, d₂, and d₃ in FIG. 12. These echoes can be interpreted as information about the distribution of the microbubbles 82 within tissue 70; by comparing the distribution of microbubbles 82 over time (comparisons can be made to the distribution of microbubbles 82 prior to any ablation, at an earlier time during ablation, or to a composite or average thereof), the progress of the formation of lesion L can be monitored. Further, by changing or sweeping the narrowband frequency (e.g., by using a frequency ramped narrowband signal, as discussed above), one can scan for a variety of sizes of microbubbles 82.

In general, higher temperatures cause more microbubbles, and longer ablation times also cause some combining and growth of preexisting microbubbles. Thus, a bubble state, with known ablation power conditions, provides information relating to thermal exposure versus depth. Via bench calibration, this information relating to thermal exposure versus depth can be interpreted as a degree of lesioning versus depth.

The foregoing approaches to measuring lesion formation can be carried out, either singly or in any combination, by a processor incorporated into control unit 130. As used herein, the term processor refers to not only a single central processing unit (“CPU”), but also to a plurality of processing units, commonly referred to as a parallel processing environment. It should also be understood that the methods disclosed herein can be hardware and/or software implemented.

Although several embodiments of this invention have been described above with a certain degree of particularity, those skilled in the art could make numerous alterations to the disclosed embodiments without departing from the spirit or scope of this invention.

For example, although the description above relates to providing a practitioner with feedback regarding lesion formation, the lesion formation information can also be used to automatically control the ablation process (e.g., control unit 130 can be programmed to discontinue the application of ablative energy when lesion L reaches a desired depth d).

As another example, in addition or as an alternative to tracking the actual and/or apparent movement of acoustic reflectors along beam path 34, their lateral (i.e., across beam path 34) cross-over frequency as they move through beam path 34 can be tracked as a measure of lesion formation by analogy to the tissue elasticity and tissue shrinkage methodologies disclosed herein.

As still another example, the several lesion monitoring methodologies discussed herein can not only be employed singly, but also in various combinations and/or weighted combinations.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of measuring lesion formation in a tissue, comprising:
acquiring a first echogram scanline of the tissue from an ultrasound imaging device operating at a first transmit power and a first gain, wherein the first echogram scanline comprises a first line scan of the tissue from a surface of the tissue to a depth within the tissue;
delivering an increment of ablation to the tissue;
acquiring a second echogram scanline of the tissue from the ultrasound imaging device after acquiring the first echogram scanline and delivering the increment of ablation to the tissue, wherein the second echogram scanline comprises a second scan line of the tissue from the surface of the tissue to the depth within the tissue;
identifying a region that appears darker in the second echogram scanline than in the first echogram scanline when the second echogram scanline is acquired with the ultrasound imaging device operating at the first transmit power and the first gain;
increasing a brightness of the second echogram scanline until the region that appears darker in the second echogram scanline than in the first echogram scanline appears as bright in the second echogram scanline as it appears in the first echograms scanline; and
after increasing the brightness of the second echogram scanline:
identifying a region that appears brighter in the second echogram scanline than it appears in the first echogram scanline; and
providing feedback about a lesion forming in the tissue based upon the region that appears brighter in the second echogram scanline than it appears in the first echogram scanline.

2. The method according to claim 1, wherein increasing a brightness of the second echogram scanline comprises increasing a receive gain of the ultrasound imaging device relative to the first gain prior to acquiring the second echogram scanline.

3. The method according to claim 1, wherein increasing a brightness of the second echogram scanline comprises increasing transmit power of the ultrasound imaging device relative to the first transmit power prior to acquiring the second echogram scanline.

4. The method according to claim 1, wherein providing feedback about a lesion forming in the tissue based upon the region that appears brighter in the second echogram scanline than it appears in the first echogram scanline comprises providing lesion depth information according to a depth of the region that appears brighter in the second echogram scanline than it appears in the first echogram scanline.

5. The method according to claim 1, wherein the step of acquiring a first echogram scanline of the tissue and the step of acquiring a second echogram scanline of the tissue occur at a common time point in a cardiac cycle and with the ultrasound imaging device at a common orientation relative to the tissue.

6. The method according to claim 1, wherein the first echogram scanline and the second echogram scanline each comprises an A-line echogram of a desired scan line through the tissue.

7. A method of measuring lesion formation in cardiac tissue, comprising:
acquiring a first A-line scan echogram of the cardiac tissue at a first cardiac deformation state;
acquiring a second A-line scan echogram of the cardiac tissue at a second cardiac deformation state;
computing a baseline elasticity of the cardiac tissue from the first A-line scan echogram and the second A-line scan echogram;
ablating the cardiac tissue; and, after ablating the cardiac tissue:
acquiring a third A-line scan echogram of the cardiac tissue at the first cardiac deformation state;
acquiring a fourth A-line scan echogram of the cardiac tissue at the second cardiac dethrmation state;
computing a revised elasticity of the cardiac tissue from the third A-line scan echogram and the fourth A-line scan echogram; and
providing feedback about a lesion forming in the cardiac tissue based upon the revised elasticity of the cardiac tissue.

8. The method according to claim 7, wherein providing feedback about a lesion forming in the cardiac tissue based upon the revised elasticity of the cardiac tissue comprises providing feedback about a lesion forming in the cardiac tissue based upon a comparison of the revised elasticity to the baseline elasticity.

9. The method according to claim 7, wherein providing feedback about a lesion forming in the cardiac tissue based upon the revised elasticity of the cardiac tissue comprises providing feedback about a lesion forming in the cardiac tissue based upon a comparison of the revised elasticity to a desired elasticity.

10. The method according to claim 7, wherein:
computing a baseline elasticity of the cardiac tissue comprises computing a baseline elasticity of the cardiac tissue based upon movement of acoustic reflectors within the cardiac tissue between the first A-line scan echogram and the second A-line scan echogram; and
computing a revised elasticity of the cardiac tissue comprises computing a revised elasticity of the cardiac tissue based upon movement of acoustic reflectors within the cardiac tissue between the third A-line scan echogram and the fourth A-line scan echogram.

11. A method of measuring lesion formation in cardiac tissue, comprising:
acquiring a first A-line scan echogram of the cardiac tissue;
ablating the cardiac tissue; and, after ablating the cardiac tissue:
acquiring a second A-line scan echogram of the cardiac tissue along a common tissue path relative to the first A-line scan echogram of the cardiac tissue;
determining an apparent shrinkage of the cardiac tissue along the A-line from the first A-line scan echogram and the second A-line scan echogram; and
providing feedback about a lesion forming in the cardiac tissue based upon the apparent shrinkage of the cardiac tissue.

12. The method according to claim 11, wherein the first A-line scan echogram and the second A-line scan echogram are each acquired with the cardiac tissue in a first cardiac deformation state.

13. The method according to claim 11, wherein determining an apparent shrinkage of the cardiac tissue from the first A-line scan echogram and the second A-line scan echogram comprises determining an apparent shrinkage based upon apparent movement of acoustic reflectors within the cardiac tissue between the first A-line scan echogram and the second A-line scan echogram.

14. A method of measuring lesion formation in cardiac tissue, comprising:
acquiring a first A-line scan echogram of the cardiac tissue at a first cardiac deformation state;
ablating the cardiac tissue; and, after ablating the cardiac tissue:
acquiring a second A-line scan echogram of the cardiac tissue at the first cardiac deformation state; and
providing feedback about a lesion forming in the cardiac tissue by analyzing at least one of actual movement of acoustic reflectors within the cardiac tissue and apparent movement of acoustic reflectors within the cardiac tissue using at least the first A-line scan and the second A-line scan.

15. The method according to claim 14, wherein providing feedback about a lesion forming in the cardiac tissue comprises providing feedback about the lesion forming in the cardiac tissue using apparent movement of acoustic reflectors due to increases in acoustic velocity within the cardiac tissue resulting from a lesion.

16. The method according to claim 14, wherein providing feedback about a lesion forming in the cardiac tissue comprises providing feedback about the lesion forming in the cardiac tissue using actual movement of acoustic reflectors due to decreases in tissue elasticity within the cardiac tissue resulting from a lesion.

17. A method of measuring lesion formation in a tissue, comprising:
emitting narrowband pulsed acoustic energy towards the tissue at a preset frequency, wherein the preset frequency comprises a resonant frequency of a microbubble characteristic of lesion formation;
detecting echoes of the emitted acoustic energy at one or more of the resonant frequency and harmonics of the resonant frequency; and providing feedback about a lesion forming in the tissue by analyzing a distribution of the microbubble characteristic of lesion formation within the tissue using the detected echoes of the emitted acoustic energy.

18. The method according to claim 17, further comprising:
ablating the tissue; and, after ablating the tissue, repeating the steps of:
emitting narrowband pulsed acoustic energy towards the tissue at a preset frequency;
detecting echoes of the emitted acoustic energy; and
analyzing a distribution of the microbubble characteristic of lesion formation within the tissue using the detected echoes of the emitted acoustic energy,
wherein providing feedback about a lesion forming in the tissue comprises analyzing a change in the distribution of the microbubble characteristic of lesion formation within the tissue from prior to ablating the tissue to after ablating the tissue.

19. A system for measuring lesion formation in a tissue, comprising:
a lesion analysis processor programmed to receive as input at least two A-line scan echograms of the tissue, to determine progress of a lesion forming in the tissue from the at least two A-line scan echograms, and to output feedback about the lesion,
wherein the at least two A-line scan echograms of the tissue are taken at common cardiac deformation states and along common scan lines.

20. The system according to claim 19, wherein the lesion analysis processor is programmed to determine progress of a lesion forming in the tissue from the at least two A-line scan echograms by comparing a brightness of a first echogram of the at least two A-line scan echograms to a brightness of a second echogram of the at least two A-line scan echograms.

21. The system according to claim 19, wherein the lesion analysis processor is programmed to determine progress of a lesion forming in the tissue from the at least two A-line scan echograms by analyzing changes in actual movement of acoustic reflectors within the tissue due to changes in tissue elasticity due to lesion formation using the at least two A-line scan echograms.

22. The system according to claim 19, wherein the lesion analysis processor is programmed to determine progress of a lesion forming in the tissue from the at least two A-line scan echograms by analyzing apparent movement of acoustic reflectors within the tissue due to increases in acoustic velocity resulting from lesion formation using the at least two A-line scan echograms.

23. The system according to claim 19, wherein the lesion analysis processor is programmed to determine progress of a lesion forming in the tissue from the at least two A-line scan echograms by analyzing changes in resonant microbubble distribution due to lesion formation using the at least two A-line scan echograms.
